(19) United States
(12) Patent Application Publication
(10) Pub. No.: US 2004/0029977 A1
(43) Pub. Date: Feb. 12, 2004

(54) Fine-grained emulsions

(76) Inventors: Rolf Kawa, Monheim (DE); Achim Ansmann, Erkrath (DE); Rainer Eskuchen, Langenfeld (DE)

Correspondence Address: COGNIS CORPORATION, PATENT DEPARTMENT, 300 BROOKSIDE AVENUE, AMBLER, PA, 19002, US

(21) Appl. No.: 10/433,114

(22) PCT Filed: Nov. 21, 2001
(86) PCT No.: PCT/EP01/13482

(30) Foreign Application Priority Data
Nov. 30, 2000 (DE) 100 59 430.1

(51) Int. Cl. A61K 47/00

(52) U.S. Cl. 514/786

(57) ABSTRACT

A process for making an emulsion having a particle size of from about 0.1 to 5 μm involving: (a) providing an oil component having a polarity of up to 5 Debye; (b) providing an emulsifier; (c) providing water; and (d) homogenizing (a)-(c), under pressure, to form the emulsion.

### FIELD OF THE INVENTION

This invention relates to a process for the production of emulsions of a certain particle size by high-pressure homogenization and to the use of these emulsions in cosmetic and/or pharmaceutical preparations.

### PRIOR ART

Emulsions with small particle sizes are growing in importance in their use in cosmetic and pharmaceutical preparations. This is attributable to their excellent performance properties, phase-stable systems being present even at very low viscosities ot <10 mPas.

For the small-particle or small-droplet emulsions known from the prior art, the choice of the emulsifier is critical. At present, fine-particle stable emulsions can only be obtained by the phase inversion method, the emulsifier structures used being exclusively structures containing ethylene oxide.

Accordingly, the problem addressed by the present invention was to provide fine-particle emulsions which could be produced irrespective of the choice of emulsifier and which, in addition, would be characterized by particular phase stability and low viscosity and would not have to be produced by the phase inversion method.

### DESCRIPTION OF THE INVENTION

The present invention relates to a process for the production of emulsions having a particle size of 0.1 to 5 μm in which oil components with a polarity of at most 5 Debye are mixed with emulsifiers and water and the resulting mixture is then homogenized under pressure. The present invention also relates to the use of emulsions with a particle size of 0.1 to 5 μm obtained by mixing oil components having a polarity of at most 5 Debye with emulsifiers and water and then homogenizing the resulting mixture under pressure in cosmetic and/or pharmaceutical preparations.

It has surprisingly been found that low-viscosity emulsions characterized by phase stability and particular particle fineness can be obtained by subjecting oil components of a certain polarity to high-pressure homogenization with emulsifiers and water. The invention includes the observation that these fine-particle emulsions can thus be obtained irrespective of the emulsifier structure used. It is a particular advantage that, in contrast to phase inversion technology, the emulsifiers used do not have to contain ethylene oxide to obtain particularly fine-particle emulsions.

Oil Components

According to the present invention, the oil components to be emulsified are characterized in that they have a polarity below 5 Debye, preferably 1.3 to 4.5 Debye and more particularly 2.5 to 4 Debye. The dipole moment of the substances in question can be calculated, for example, using the Onsager formula [cf. Seifen-Öle-Fette-Wachse, 115, 459-61 (1989)], the necessary material criteria, such as refraction and molecular weight, being known from the literature or being able to be determined by the usual physical/chemical methods. Suitable oil components according to the invention are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkyl hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-/di-/triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates based on C₆₋₁₈ and preferably C₈₋₁₀ fatty alcohols, diethylhexyl naphthalates (HallBrite TQ), esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils (cyclomethicone, silicon methicones, etc.) and/or aliphatic or naphthenic hydrocarbons such as, for example, mineral oil, Vaseline, squalane, squalene or dialkyl cyclohexanes.

The oil components according to the invention may be used in quantities of 1 to 70, preferably 10 to 50 and more particularly 15 to 30% by weight, based on the final composition, in the process according to the invention.

Emulsifiers

According to the invention, suitable emulsifiers or dispersants are those which, in combination with the oils of defined polarity characterized above, cover the phase interfaces particularly quickly:

products of the addition of 2 to 30 mol ethylene oxide and/or 0 to 5 mol propylene oxide onto linear C₈₋₂₂ fatty alcohols, C₁₂₋₂₂ fatty acids, alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alk(en)yl group and ethoxylated analogs thereof;

products of the addition of 1 to 15 mol ethylene oxide with castor oil and/or hydrogenated castor oil;

products of the addition of 15 to 60 mol ethylene oxide with castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof with 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof with 1 to 30 mol ethylene oxide;

mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

protein fatty acid condensates, preferably based on wheat protein;

wool wax alcohols,

polysiloxane/polyalkyl/polyether copolymers and corresponding derivatives,

block copolymers, for example Polyethyleneglycol-30 Dipolyhydroxy-stearate;

polymer emulsifiers, for example Pemulen types (TR-1, TR-2) from Goodrich;

polyalkylene glycols and

glycerol carbonate.

Particularly preferred emulsifiers are, for example, Cetyl Dimethicone Copolyol (for example Abil EM-90), Polyglyceryl-2 Dipolyhydroxystearate (for example Dehymuls PGPH), Polyglycerin-3-Diisostearate (for example Lameform TGI), Polyglyceryl-4 Isostearate (for example Isolan GI 34), Polyglyceryl-3 Oleate, Diisostearoyl Polyglyceryl-3 Diisostearate (for example Isolan PDI), Polyglyceryl-3 Methylglucose Distearate (for example Tego Care 450), Polyglyceryl-3 Beeswax (for example Cera Bellina), Polyglyceryl-4 Caprate (for example Polyglycerol Caprate T2010/90), Polyglyceryl-3 Cetyl Ether (for example Chimexane NL), Polyglyceryl-3 Distearate (for example Cremophor GS 32) and Polyglyceryl Polyricinoleate (for example Admul WOL 1403), Glyceryl Oleate (for example Monomuls 90-O 18), Alkyl Glucoside (for example Plantacare 1200, Emulgade PL 68/50, Montanov 68, Tego Care CG 90, Tego Glucosid L 55), Methyl Glucose Isostearate (for example Tego Care IS), Methyl Glucose Sesquistearate (Tego Care PS), Sodium Cocoyl Hydrolyzed Wheat Protein (for example Gluadin WK), Potassium Cetyl Phosphate (for example Amphisol K, Crodafos CKP), Sodium Alkylsulfate (for example Lanette E), Sucrose Ester (for example Crodesta F-10, F-20, F-50, F-70, F-110, F-160, SL-40), ethoxylated and/or propoxylated fatty alcohols, fatty acids, castor oils or hydrogenated castor oils (for example Eumulgin B2, B2, B3, L, HRE 40, HRE 60, RO 40, Cremophor HRE 40, HRE 60, L, WO 7, Dehymuls HRE 7, Arlacel 989), PEG-30 Dipolyhydroxystearate, Sorbitan Ester, Sorbitan Ester ethoxylated and/or propoxylated and mixtures thereof. A particularly effective mixture consists of Polyglyceryl-2 Dipolyhydroxystearate and Lauryl Glucoside and Glycerin (for example Eumulgin VL 75).

The emulsifiers according to the invention may be used in quantities of 0.1 to 20, preferably 1 to 10 and more particularly 3 to 7% by weight, based on the preparations, in the process according to the invention.

High-Pressure Homogenization

In the emulsification art, homogenization is understood to be the very fine size reduction of the disperse phase of a crude emulsion. In this process, the particle size spectrum of the crude emulsion is clearly displaced towards smaller droplets. The size reduction of the droplets results in the formation of new phase interfaces which have to be rapidly covered completely by emulsifier molecules so that the new droplets formed are better stabilized and can be further size-reduced more easily by virtue of the low interfacial tension. A particular form of homogenization is high-pressure homogenization where the droplets are size-reduced by the input of mechanical energy in the form of a differential pressure so that new phase interfaces are rapidly formed in large numb rs.

The necessary or preferred pressure ranges for high-pressure homogenization are dependent upon the type of homogenizing valve or homogenizing nozzle used. The emulsions according to the invention are preferably homogenized by radial diffusors or counter-jet dispersers as homogenizing nozzles or micromixers.

Where radial diffusors as used as the homogenizing valve (such as, for example, flat, serrated-edge or knife-edge nozzles), pressures of 100 to 1500 bar, preferably 200 to 800 bar and more particularly 400 to 600 bar are applied.

Where counter-jet dispersers (et dispersers, microfluidizers) are used as homogenizing nozzles, typical pressures are in the range from 10 to 100 bar. In this case, the preferred pressure range is between 20 and 60 bar.

Where the emulsions are produced using micromixers, a typical pressure range is between 2 and 30 bar and preferably between 5 and 20 bar. The micromixer used (manufacturer: Institut für Mikrosystemtechnik Mainz, IMM) is a “static mixer” with a channel width of 25μ. In view of the narrow channels, two liquid phases are mixed by diffusion. Micromixers have the advantage at low pressures of producing fine-particle emulsions with narrow particle size distributions under particularly moderate conditions.

In order to obtain fine-particle emulsions with a monomodal and narrow particle size distribution, it can be of advantage to combine various emulsifying processes with one another. For example, a “pre-emulsion” can be prepared in a stirred vessel and subsequently homogenized by dispersion in a so-called single pass through a rotor/stator homogenizer and then through a high-pressure homogenizer. By single pass is meant a procedure whereby the entire contents of a vessel are passed once through the homogenizer into another vessel. In contrast to the so-called recycle principle, this ensures that each liquid element passes through the homogenizer once, leaving behind no coarse emulsion droplets which could form the starting point for the breakup of the emulsion.

Rotor/stator systems can be such machines as toothed colloid mills or machines which consist of one or more rotors and stators with throughflow openings in the form of slits or cylindrical or rectangular holes, for example of the Cavitron, Supraton, Siefer, Bran+Lübbe, IKA, Koruma, Silverson types, etc.

The advantage of high-pressure homogenization is that small droplets with a very narrow distribution are formed very easily which is an advantage where low-viscosity phase-stable emulsions are to be produced. By virtue of the performance-related advantages of emulsions produced by high-pressure homogenization, increasing efforts are also being made in the pharmaceutical industry to employ such homogenizing techniques. Due to the fact that a new interface is formed particularly quickly, the emulsifier and carrier phase have to meet exacting requirements because the emulsifiers have to cover the interface spontaneously and very quickly to ensure optimal phase stability. It has been found that the speed with which the emulsifiers cover the interface can be significantly increased if the oil components to be emulsified have a polarity beow 5 Debye. The dipole moment of the substances in question can be calculated, for example, using Onsager's formula [cf. Seifen-Öle-Fette-Wachse, 115, 459-61 (1989)].

Cosmetic Preparations

Particularly fine-particle emulsions with a narrow particle size distribution are obtained by the process according to the invention. A preferred emulsion has the following composition:

(a) 1 to 70, preferably 10 to 50 and more particularly 15 to 30% by weight of oil components with a polarity of at most 5 Debye,

(b) 0.1 to 20, preferably 1 to 10 and more particularly 3 to 7% by weight of emulsifiers and

(c) 10 to 90, preferably 20 to 70 and more particularly 30 to 50% by weight of water.

The emulsions produced by the process according to the invention have particle sizes of 0.1 to 5, preferably 1.4 to 4 and more particularly 1.8 to 3 μm. A diffraction pattern is determined by laser diffraction. The particle size distribution is then calculated from the line intensities of the diffraction pattern using the Frauhofer theory in conjunction with EDP.

The emulsions may contain UV protection factors, mild surfactants, pearlizing waxes, consistency factors, thickeners, superfatting agents, stabilizers, polymers, silicone compounds, fats, waxes, lecithins, phospholipids, biogenic agents, antioxidants, deodorants, antiperspirants, antidandruff agents, swelling agents, insect repellents, self-tanning agents, tyrosine inhibitors (depigmenting agents), hydrotropes, solubilizers, preservatives, perfume oils, dyes and the like as further auxiliaries and additives.

UV Protection Factors

UV protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP 0693471 B1;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzo-phenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 19712033 A1 (BASF). The UV-A and UV-B filters may of course also be used in the form of mixtures. Particularly favorable combinations consist of the derivatives of benzoyl methane, for example 4-tert.butyl-4′-methoxydibenzoyl methane (Parsol® 1789) and 2-cyano-3,3-phenylcinnamic acid-2-ethylhexyl ester (Octocrylene), in combination with esters of cinnamic acid, preferably 4-methoxycinnamic acid-2-ethylhexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used. Other suitable UV filters can be found in P. Finkel's review in SÖFW-Journal 122, 543 (1996) and in Parf. Kosm. 3, 11 (1999).

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution.

Waxes

Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs) and correspond to the following general formula:

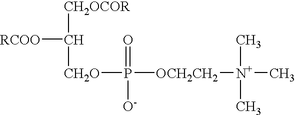

where R typically represents linear aliphatic hydrocarbon radicals containing 15 to 17 carbon atoms and up to 4 cis-double bonds. Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially coconuffatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols®) and Pemulen types [Goodrich]; Synthalens® [Sigma]; Keltrol types [Kelco]; Sepigel types [Seppic]; Salcare types [Allied Colloids]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacryl-amide/methyl methacrylate/tert.-butylaminoethyl methacrylate/2-hydroxy-propyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosmetics & Toiletries, Vol. 108, May 1993, pages 95 et seq.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Antioxidants

Antioxidants which interrupt the photochemical reaction chain which is initiated when UV rays penetrate into the skin may also be added. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearyl-thiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmol to μmol/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxy-toluene, butyl hydroxyanisole, nordihydroguaiac resin acid, nordihydro-guaiaretic acid, trihydroxybutyrophenone, uric acid and derivatives thereof, mannose and derivatives thereof, Superoxid-Dismutase, zinc and derivatives thereof (for example ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

In the context of the invention, biogenic agents are, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Self-Tanning Agents and Deligmenting Agents

A suitable self-tanning agent is dihydroxyacetone. Suitable tyrosine inhibitors which prevent the formation of melanin and are used in depigmenting agents are, for example, arbutin, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipenta-erythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetivert oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Dyes

Suitable dyes are any of the substances suitable and approved for cosmetic purposes as listed, for example, in the publication “Kosmetische Fäirbemittel” of the Farbstoffkommission der Deutschen Forschungs-gemeinschaft, Verlag Chemie, Weinheim, 1984, pages 81 to 106. These dyes are normally used in concentrations of 0.001 to 0.1% by weight, based on the mixture as a whole.

Fillers

Both organic and inorganic fillers may be used. Talcum, mica (for example sericite), barium sulfate, polyethylenes, polytetrafluroethylenes, nylon powder and polymethyl methacrylate powder (PMMA) are preferably used.

The total percentage content of auxiliaries and additives may be from 1 to 80% by weight and is preferably from 5 to 50% by weight and more particularly from 7 to 10% by weight, based on the preparation. The preparations may be produced by standard hot or cold emulsification processes.

### EXAMPLES

Test Formulation:

| | |
|---|---|
| Oil component | 16.0% by weight |
| Emulsifier | 1.0 or 4.5% by weight |
| Water | to 100% by weight |

| Oil component: | Emulsifier: |
|---|---|
| 1. Dicaprylyl Carbonate (1.5 Debye) | Ceteareth-20* |
| 2. Cocoglycerides (2.5 Debye | Eumulgin VL 75 (INCI see text)** |
| 3. Castor oil (4.2 Debye) | |
| 4. Myreth-3 Myristate (5.5 Debye) | |

*1.0% by weight
**45% by weight
Oils 1 to 3 correspond to the invention, oil 4 is intended for comparison.

Evaluation Criteria:

In order to determine particle size, a diffraction pattern is established by laser diffraction. The particle size distribution is then calculated from the light intensities of the diffraction patterns using the Frauhofer theory (Sympatec Helos)

immediately after production

after 2 weeks at 40° C.

I. Process (According to the Invention):

preparation of a pre-emulsion in a stirred vessel

homogenization of the pre-emulsion in the rotor/stator homogenizing system (single pass)

high-pressure homogenization by a LAB 60 (APV Gaulin)

homogenizing valve: radial diffusor (flat nozzle)

pressure: 500 bar

Results:

A. Particle size immediately after Droduction as the x90 value. i.e. 90%
of the particles are smaller than

| Ceteareth-20 | 1.0% by weight | | |
|---|---|---|---|
| Dicaprylyl Carbonate | 1.7 μm | | Viscosity |
| Cocoglycerides | 1.4 μm | {close oversize brace} | (Höppler, ball 1, 20° C.): |
| Castor Oil | 2.9 μm | | 4.5 mPas |
| Myreth-3 Myristate | no emulsion formed (no storage tests) | | |
| Eumulgin VL 75 | 4.5% by weight | | |
| Dicaprylyl Carbonate | 1.8 μm | | Viscosity |
| Cocoglycerides | 2.1 μm | {close oversize brace} | (Höppler, bail 1, 20° C.): |
| Castor Oil | 2.7 μm | | 2 mPas |

| | |
|---|---|
| Myreth-3 Myristate | no emulsion formed (no storage tests) |

B. Particle size after 2 weeks at 40° C. as the x90 value, i.e. 90% of the
particles are smaller than

| Ceteareth-20 | 1.0% by weight | | |
|---|---|---|---|
| Dicaprylyl Carbonate | 1.5 μm | | Viscosity |
| Cocoglycerides | 1.4 μm | {close oversize brace} | (Höppler, ball 1, 20° C.): |
| Castor Oil | 3.2 μm | | 45 mPas |
| Eumulpin VL 75 | 4.5% by weight | | |
| Dicaprylyl Carbonate | 3.8 μm | | Viscosity |
| Cocoglycerides | 2.3 μm | {close oversize brace} | (Höppler, ball 1, 20° C.): |
| Castor Oil | 4.5 μm | | 2 mPas |

In the case of the oils according to the invention, no phase separation was observed.

II. Known Process for Comparison:

Preparation of a pre-emulsion in a stirred vessel

Homogenization of the pre-emulsion in a rotor/stator homogenizing system (single pass)

Result:

A. Particle size immediately after production as the x90 value, i.e. 90%
of the particles are smaller than

| Ceteareth-20 | 1.0% by weight | | |
|---|---|---|---|
| Dicaprylyl Carbonate | 9 μm | | Viscosity |
| Cocoglycerides | 10 μm | {close oversize brace} | (Höppler, ball 1, 20° C.): |
| Castor Oil | 15 μm | | 6.8 mPas |
| Myreth-3 Myristate | no emulsion formed (no storage tests) | | |
| Eumulgin VL 75 | 4.5% by weight | | |
| Dicaprylyl Carbonate | 11 μm | | Viscosity |
| Cocoglycerides | 8 μm | {close oversize brace} | (Höppler, ball 1, 20° C.): |
| Castor Oil | 14 μm | | 2.5 mPas |

| | |
|---|---|
| Myreth-3 Myristate | no emulsion formed (no storage tests) |

Particle size could not be determined after sorage at 40° C. because the emulsions had separated after only 24 h.

1. A process for the production of emulsions with a particle size of 0.1 to 5 μm, in which oil components with a polarity of at most 5 Debye are mixed with emulsifiers and water and the resulting mixture is homogenized under pressure.

2. A process as claimed in claim 1, characterized in that the oil components used are selected from the group consisting of Guerbet alcohols based on fatty alcohols containing 6 to 18 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols, esters of linear C₆₋₂₂ fatty acids with branched alcohols, esters of C₁₈₋₃₈ alkyl hydroxycarboxylic acids with linear or branched C₆₋₂₂ fatty alcohols, esters of linear and/or branched fatty acids with polyhydric alcohols and/or Guerbet alcohols, triglycerides based on C₆₋₁₀ fatty acids, liquid mono-/di-/triglyceride mixtures based on C₆₋₁₈ fatty acids, esters of C₆₋₂₂ fatty alcohols and/or Guerbet alcohols with aromatic carboxylic acids, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates based on C₆₋₁₈ fatty alcohols, diethylhexyl naphthalates, esters of benzoic acid with linear and/or branched C₆₋₂₂ alcohols, linear or branched, symmetrical or nonsymmetrical dialkyl ethers containing 6 to 22 carbon atoms per alkyl group, ring opening products of epoxidized fatty acid esters with polyols, silicone oils and/or aliphatic or naphthenic hydrocarbons.

3. A process as claimed in claims 1 and/or 2, characterized in that the emulsifiers used are selected from the group consisting of Cetyl Dimethicone Copolyol, Polyglyceryl-2 Dipolyhydroxystearate, Polyglycerin-3-Diisostearate, Polyglyceryl-4 Isostearate, Polyglyceryl-3 Oleate, Diisostearoyl Polyglyceryl-3 Diisostearate, Polyglyceryl-3 Methylglucose Distearate, Polyglyceryl-3 Beeswax, Polyglyceryl-4 Caprate, Polyglyceryl-3 Cetyl Ether, Polyglyceryl-3 Distearate and Polyglyceryl Polyricinoleate, Glyceryl Oleate, Alkyl, Methyl Glucose Isostearate, Methyl Glucose Sesquistearate, Sodium Cocoyl Hydrolyzed Wheat Protein, Potassium Cetyl Phosphate, Sodium Alkylsulfate, Sucrose Ester, ethoxylated and/or propoxylated fatty alcohols, fatty acids, castor oils or hydrogenated castor oils, PEG-30 Dipolyhydroxystearate, Sorbitan Ester, Sorbitan Ester ethoxylated and/or propoxylated and mixtures thereof.

4. A process as claimed in at least one of claims 1 to 3, characterized in that homogenization is carried out under pressures of 2 to 1,500 bar.

5. A process as claimed in at least one of claims 1 to 4, characterized in that homogenization is carried out by radial diffusors or counter-jet dispersers as homogenizing nozzles or micromixers.

6. A process as claimed in at least one of claims 1 to 5, characterized in that homogenization is carried out by radial diffusors as homogenizing nozzles under pressures of 100 to 1,500 bar.

7. A process as claimed in at least one of claims 1 to 6, characterized in that homogenization is carried out by counter-jet dispersers as homogenizing nozzles under pressures of 10 to 100 bar.

8. A process as claimed in at least one of claims 1 to 7, characterized in that homogenization is carried out by micromixers under pressures of 2 to 30 bar.

9. A process as claimed in at least one of claims 1 to 8, characterized in that the high-pressure homogenization is preceded by dispersion with a rotor/stator homogenizer.

10. The use of emulsions with a particle size of 0.1 to 5 μm obtained by mixing oil components having a polarity of at most 5 Debye with emulsifiers and water and homogenizing the resulting mixture under pressure in cosmetic and/or pharmaceutical preparations.
